(19) United States
(12) Patent Application Publication
(10) Pub. No.: US 2026/0209145 A1
(43) Pub. Date: Jul. 23, 2026

(54) CHROMIUM ALUMINA CATALYSTS FOR PARAFFIN DEHYDROGENATION

(71) Applicant: SABIC GLOBAL TECHNOLOGIES B.V., Bergen op Zoom (NL)

(72) Inventors: Biju Maippan Devassy, Bangalore (IN); Vinod Sankaran Nair, Bangalore (IN); Velayutham Saravanan, Hosur (IN); Mahabala PS, Bangalore (IN)

(21) Appl. No.: 19/140,932

(22) PCT Filed: Dec. 18, 2023
(86) PCT No.: PCT/EP2023/086282

§ 371 Date: Jun. 18, 2025

Related U.S. Application Data
(60) Provisional application No. 63/476,644, filed on Dec. 22, 2022.

(51) Int. Cl. C07C 5/333; B01J 23/26; B01J 37/02; B01J 37/08

(52) U.S. Cl. CPC C07C 5/3332; B01J 23/26; B01J 37/0207; B01J 37/0236; B01J 37/08; B01J 2523/31; B01J 2523/67

(57) ABSTRACT

Methods of preparing and using alkane dehydrogenation catalysts are provided. The method for making an alkane dehydrogenation catalyst may include providing an alumina support derived from a plurality of aluminium hydroxides, contacting the alumina support with a water-soluble chromium source and a metal oxide source to provide an impregnated alumina support, and drying and calcining the impregnated alumina support to produce an alkane dehydrogenation catalyst. The catalyst can contain about 60 wt. % to about 95 wt. % of alumina, about 5 wt. % to about 40 wt. % of trivalent chromium oxide, and about 0.1 wt. % to about 5 wt. % of the alkali metal oxide.

### CROSS REFERENCE TO RELATED APPLICATIONS

This application claims priority to and the benefit of the filing date of European Patent Application No. 63476644, filed Dec. 22, 2022, which is incorporated herein by reference in its entirety.

### TECHNICAL FIELD

The present disclosure generally relates to methods for preparing and using an alkane dehydrogenation catalyst. More specifically, the present disclosure relates to, among other embodiments, methods for the use and preparation of chromia-alumina dehydrogenation catalysts for the dehydrogenation of paraffins.

### BACKGROUND

Alkane dehydrogenation is a recognized process for the production of a variety of useful hydrocarbon products, such as isobutylene for conversion to MTBE, as well as isooctane and alkylates to supplement and enrich gasolines. There are several current catalytic processes useful for the catalytic dehydrogenation of light alkanes, including the Süd-Chemie CATOFIN® process, UOP's Oleflex® process, Phillips' Star™ process, and the Snamprogetti-Yarsintee process. The catalysts that are used in these processes are manufactured from two different groups of materials. The Süd-Chemie CATOFIN® process and the Snamprogetti-Yarsintee process utilize chromia-alumina catalysts. In contrast, the catalysts for the UOP and Phillips processes include supported precious metal platinum as catalysts.

Chromia-alumina dehydrogenation catalyst technology has been in use for many decades. The stability of dehydrogenation catalysts plays an important role in the overall efficiency of the dehydrogenation process. Because of the extreme temperature ranges at which the catalytic dehydrogenation procedure is conducted, the life expectancy of the catalyst is often limited. Thus, improving the stability of the catalyst translates into longer catalyst life, allowing for better catalyst utilization and ultimately resulting in lower consumption of the catalyst during the dehydrogenation process. One of the important requirements in the production of pelleted/extruded catalysts for use in fixed-bed catalytic operations is that the catalyst be of sufficient strength to support its own weight as well as the process-stream turbulence and similar disturbances ordinarily encountered in fixed-bed processes. Failure to satisfy these requirements is a frequent source of difficulty. Additionally, the gradual loss of mechanical strength during use is a common occurrence necessitating the discarding of catalyst which in other respects would continue to be entirely satisfactory. Accordingly, the Applicant has recognized that there exists a need for a simple, cost effective method for making a chromia-alumina dehydrogenation catalyst, and which exhibits good activity, improved stability, and suitable mechanical strength for the dehydrogenation of paraffins.

### SUMMARY OF THE INVENTION

To address shortcomings in the art, the Applicant has developed methods for the preparation and use of an alkane dehydrogenation catalyst. Embodiments of methods for making an alkane dehydrogenation catalyst include the steps of providing a transition alumina support derived from a plurality of aluminium hydroxides, the plurality of aluminium hydroxides containing about 85 weight percent (wt. %) to about 99 wt. % of crystalline aluminium trihydroxide and about 1 wt. % to about 15 wt. % of crystalline aluminium oxide-hydroxide or gelatinous aluminium hydroxide or combinations thereof. The method further includes the steps of contacting the alumina support with a water-soluble chromium(III) oxide source and an alkali metal oxide source to provide an impregnated alumina support and drying and calcining the impregnated alumina support to produce an alkane dehydrogenation catalyst containing about 60 wt. % to about 95 wt. % of alumina, about 5 wt. % to about 40 wt. % of the chromium(III) oxide, and about 0.1 wt. % to about 5 wt. % of the alkali metal oxide. In certain embodiments, the transition alumina support is substantially an eta-alumina. The impregnated alumina support is calcined at a temperature ranging from about 700 degrees Celsius (° C.) to 800° C.

The alkane dehydrogenation catalyst can contain about 60 wt. % to about 90 wt. % of alumina, about 10 wt. % to about 40 wt. % of the chromium(III) oxide, and about 0.1 wt. % to about 5 wt. % of the alkali metal oxide. The crystalline aluminium trihydroxide can contain one or more of bayerite and nordstrandite. The crystalline aluminium oxide-hydroxide can contain boehmite. The gelatinous aluminium hydroxides can contain one or more of amorphous aluminium hydroxide and pseudoboehmite.

Embodiments disclosed here also include methods for using an alkane dehydrogenation catalyst. One such method for dehydrogenation of an alkane includes the steps of loading a reactor with a dehydrogenation catalyst prepared as disclosed herein and supplying a feed containing an alkane through the reactor at a temperature sufficient to dehydrogenate the alkane and produce an olefin. In certain embodiments, the temperature sufficient to dehydrogenate the alkane ranges from 400° C. to 800° C.

Still other aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description. Furthermore, it is to be understood that the features of the various embodiments described herein are not mutually exclusive and may exist in various combinations and permutations.

### DETAILED DESCRIPTION OF THE INVENTION

The present disclosure describes various embodiments related to methods for preparing and using an alkane dehydrogenation catalyst. Further embodiments may be described and disclosed.

In the following description, numerous details are set forth in order to provide a thorough understanding of the various embodiments. In other instances, well-known processes, devices, and systems may not have been described in particular detail to not unnecessarily obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” is defined as being close to as understood by one of ordinary skill in the art. In one non-limiting embodiment, the terms are defined to be within 10%, preferably within 5%, more preferably within 1%, and most preferably within 0.5%.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.”

The terms “wt. %0”, “vol. %”, or “mol. %” refer to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Embodiments of methods for making an alkane dehydrogenation catalyst include the steps of providing a transition alumina support derived from a plurality of aluminium hydroxides, the plurality of aluminium hydroxides containing about 85 wt. % to about 99 wt. % of crystalline aluminium trihydroxide and about 1 wt. % to about 15 wt. % of crystalline aluminium oxide-hydroxide or gelatinous aluminium hydroxide or combinations thereof. The method further includes the steps of contacting the alumina support with a water-soluble chromium(III) oxide source and an alkali metal oxide source to provide an impregnated alumina support and drying and calcining the impregnated alumina support to produce an alkane dehydrogenation catalyst containing about 60 wt. % to about 95 wt. % of alumina, about 5 wt. % to about 40 wt. % of the chromium(III) oxide, and about 0.1 wt. % to about 5 wt. % of the alkali metal oxide. The impregnated alumina support is calcined at a temperature ranging from about 700° C. to 800° C. Certain embodiments of the alkane dehydrogenation catalyst contain about 60 wt. % to about 90 wt. % of alumina, about 10 wt. % to about 40 wt. % of the chromium(III) oxide, and about 0.1 wt. % to about 5 wt. % of the alkali metal oxide. These weight percentages are calculated on a calcined basis.

The transition alumina support is derived from a plurality of aluminium hydroxides and contains about 85 wt. % to about 99 wt. % of crystalline aluminium trihydroxide and about 1 wt. % to about 15 wt. % of crystalline aluminium oxide-hydroxide or gelatinous aluminium hydroxide or combinations thereof. The crystalline aluminium trihydroxide can contain one or more of bayerite and nordstrandite. The crystalline aluminium oxide-hydroxide can contain boehmite. The gelatinous aluminium hydroxide can contain one or more of amorphous aluminium hydroxide and pseudoboehmite. “Transition aluminas” are one or more aluminas other than alpha-alumina, which are capable of being at least partially converted to alpha-alumina under thermal treatment at 900° C. or greater. Transition aluminas include, but are not limited to gamma-alumina, delta-alumina, eta-alumina, kappa-alumina, chi-alumina, rho-alumina, and theta-alumina. In certain embodiments, the transition alumina support is substantially an eta-alumina. An alumina support that contains substantially eta-alumina refers to an alumina support containing from about 75 wt. % to about 99 wt. % of eta-alumina and all ranges and values therebetween, such as from about 78 wt. % to about 93 wt. %, about 80 wt. % to about 90 wt. %, about 83 wt. %, about 85 wt. %, or about 87 wt. %.

The plurality of aluminium hydroxides can contain from about 87 wt. % to about 99 wt. % of crystalline aluminium trihydroxide. The plurality of aluminium hydroxides can contain from about 90 wt. % to about 99 wt. % of crystalline aluminium trihydroxide. The plurality of aluminium hydroxides can contain from about 85 wt. % to about 97 wt. % of crystalline aluminium trihydroxide. The plurality of aluminium hydroxides can contain from about 87 wt. % to about 97 wt. % of crystalline aluminium trihydroxide. The plurality of aluminium hydroxides can contain from about 89 wt. % to about 97 wt. % of crystalline aluminium trihydroxide. The plurality of aluminium hydroxides can contain from about 85 wt. % to about 95 wt. % of crystalline aluminium trihydroxide. plurality of aluminium hydroxides can contain from about 85 wt. % to about 92 wt. % of crystalline aluminium trihydroxide. In certain embodiments, the crystalline aluminium trihydroxide contains bayerite, nordstrandite, or both. Calcination of crystalline bayerite leads to destruction of the original highly crystalline framework and evolves through alumina phases eta and theta before reaching alpha alumina.

The plurality of aluminium hydroxides can contain from about 1 wt. % to about 15 wt. % of crystalline aluminium oxide-hydroxide or gelatinous aluminium hydroxide or combinations thereof. The plurality of aluminium hydroxides can contain from about 3 wt. % to about 15 wt. % of crystalline aluminium oxide-hydroxide or gelatinous aluminium hydroxide or combinations thereof. The plurality of aluminium hydroxides can contain from about 5 wt. % to about 15 wt. % of crystalline aluminium oxide-hydroxide or gelatinous aluminium hydroxide or combinations thereof. The plurality of aluminium hydroxides can contain from about 7 wt. % to about 15 wt. % of crystalline aluminium oxide-hydroxide or gelatinous aluminium hydroxide or combinations thereof. The plurality of aluminium hydroxides can contain from about 9 wt. % to about 15 wt. % of crystalline aluminium oxide-hydroxide or gelatinous aluminium hydroxide or combinations thereof. The plurality of aluminium hydroxides can contain from about 1 wt. % to about 13 wt. % of crystalline aluminium oxide-hydroxide or gelatinous aluminium hydroxide or combinations thereof. The plurality of aluminium hydroxides can contain from about 1 wt. % to about 11 wt. % of crystalline aluminium oxide-hydroxide or gelatinous aluminium hydroxide or combinations thereof. The plurality of aluminium hydroxides can contain from about 3 wt. % to about 13 wt. % of crystalline aluminium oxide-hydroxide or gelatinous aluminium hydroxide or combinations thereof. In certain embodiments, the crystalline aluminium oxide-hydroxide contains boehmite. In certain embodiments, the gelatinous aluminium hydroxides contains amorphous aluminium hydroxide, pseudoboehmite, or both. Calcination of boehmite, pseudoboehmite and amorphous aluminium hydroxide leads to the formation of gamma-alumina, delta-alumina, theta-alumina before reaching alpha-alumina.

In an embodiment, the alumina support used for the catalyst preparation contains eta-alumina. In other embodiments, the alumina support contains 95 wt. % eta-alumina and 5 wt. % gamma-alumina. In other embodiments, the alumina support contains 90 wt. % eta-alumina and 10 wt. % gamma-alumina. Certain embodiments of the alumina support contain 85 wt. % eta-alumina and 15 wt. % gamma-alumina.

In certain embodiments, the catalyst is in the form of extrudates having a diameter in a range of 2 to 5 mm and all ranges and values therebetween including ranges of 2 to 3 mm, 2 to 4 mm, 2.5 to 4 mm, 2.5 to 4.5 mm, and 3 to 4 mm. In certain embodiments, the plurality of aluminium hydroxides is mixed with a nitric acid and then formed into cylindrical extrudates (about 3.5 mm diameter) using an extruder. The extrudates were dried and calcined, and cooled to room temperature without external cooling.

The calcined alumina extrudates are subject to an incipient wetness technique with an impregnation solution containing a water-soluble chromium(III) oxide source and an alkali metal oxide source. In certain embodiments, the alkali metal oxide source contains an alkali metal hydroxide, an alkali metal chromate, an alkali metal dichromate, or combinations thereof. The alkali metal can be sodium, lithium, or potassium. For example, the calcined alumina extrudates are subject to an incipient wetness technique with an impregnation solution containing a chromium oxide source and sodium dichromate dihydrate. In certain embodiments, the impregnated alumina support is calcined at a temperature ranging from about 500° C. to 800° C. In certain embodiments, the impregnated alumina support is calcined at a temperature ranging from about 500° C. to 700° C. In certain embodiments, the impregnated alumina support is calcined at a temperature ranging from about 550° C. to 650° C. Embodiments of the catalysts include a catalyst with a composition of 24.24 wt. % Cr₂O₃, 0.53 wt. % Na₂O, 1.02 wt. % La₂O₃, and 74.21 wt. % Al₂O₃.

Embodiments disclosed here also include methods for using an alkane dehydrogenation catalyst. One such method for dehydrogenation of an alkane includes the steps of loading a reactor with a dehydrogenation catalyst prepared as disclosed herein and supplying a feed containing an alkane through the reactor at a temperature sufficient to dehydrogenate the alkane and produce an olefin. In certain embodiments, the temperature sufficient to dehydrogenate the alkane ranges from 400° C. to 800° C., and all ranges and values therebetween including ranges such as 400° C. to 780° C., 420° C. to 780° C., 440° C. to 760° C., 450° C. to 750° C., 500° C. to 700° C., and 600° C. to 800° C. The reaction conditions may further include a reaction pressure of 0.2 bar to 1 bar and all ranges and values therebetween including 0.3 bar, 0.4 bar, 0.5 bar, 0.6 bar, 0.7 bar, 0.8 bar, and 0.9 bar. The reaction conditions may further include a gas hourly space velocity of 300 hr⁻¹ to 800 hr⁻¹ and all ranges and values between the upper and lower limits of the range. The dehydrogenation catalysts prepared according to the presently disclosed methods have good activity as well as improved stability and mechanical strength.

The feed containing an alkane can include C2 to C20 linear, iso-, and cyclo-alkanes, which are substantially saturated compounds containing hydrogen and carbon. The feed encompasses a range of cyclic and linear alkanes, such as ethane, propane, n-butane, isobutane, n-pentane, isopentane, n-hexane, 2-methylpentane, 3-methylpentane, 2,2-dimethylbutane, n-heptane, 2-methylhexane, 2,2,3-trimethylbutane, cyclopentane, cyclohexane, methylcyclopentane, ethylcyclopentane, n-propylcyclopentane and 1,3-dimethylcyclohexane. For example, propane can be used as an alkane-containing feed, which can be dehydrogenated to produce propylene, and isobutane can be used as an alkane-containing feed, which can be dehydrogenated to produce isobutylene. In certain aspects, substantially all of the hydrocarbon in the alkane-containing feed can be a single alkane, such as pure propane or pure butane.

In certain embodiments, the temperature sufficient to dehydrogenate the alkane ranges from 400° C. to 800° C., and all ranges and values therebetween including ranges such as 400° C. to 780° C., 420° C. to 780° C., 440° C. to 760° C., 450° C. to 750° C., 500° C. to 700° C., and 600° C. to 800° C. The reaction conditions may further include a reaction pressure of 0.2 bar to 1 bar and all ranges and values therebetween including 0.3 bar, 0.4 bar, 0.5 bar, 0.6 bar, 0.7 bar, 0.8 bar, and 0.9 bar. The reaction conditions may further include a gas hourly space velocity of 300 hr⁻¹ to 800 hr¹ and all ranges and values between the upper and lower limits of the range.

Certain embodiments of the alkane dehydrogenation catalyst are based on a transition alumina support derived from substantially all eta-alumina. These catalysts have a surface area of about 88.8 square meters per gram (m²/g) and a crush strength of about 1.77 decaNewton per millimeter (daN/mm). Certain embodiments of the alkane dehydrogenation catalyst are based on a transition alumina support derived from about 95 wt. % eta-alumina and about 5 wt. % gamma-alumina. These catalysts have a surface area of about 94.9 m²/g and a crush strength of about 1.95 daN/mm. Certain embodiments of the alkane dehydrogenation catalyst are based on a transition alumina support derived from about 90 wt. % eta-alumina and 10 wt. % gamma-alumina. These catalysts have a surface area of about 92.9 m²/g and a crush strength of about 2.45 daN/mm. Certain embodiments of the alkane dehydrogenation catalyst are based on a transition alumina support derived from about 85 wt. % eta-alumina and 15 wt. % gamma-alumina. These catalysts have a surface area of about 97 m²/g and a crush strength of about 2.74 daN/mm. As discussed in detail in Example 8 and Table 1, there were similar isobutane conversion and isobutylene yields for catalysts prepared using alumina supports containing up to 15 wt. % gamma-alumina. The catalyst with the alumina support containing 20 wt. % gamma-alumina showed lower isobutylene yield and lower catalyst stability.

### EXAMPLES

Specific examples of certain embodiments are included here for illustrative purposes only and are not intended to limit any aspect of the claimed embodiments.

### Example 1

The alumina support with eta-alumina used for the catalyst preparation is prepared as follows: About 3125 grams of Bayerite (Pural BT, SASOL) is mixed for 10 minutes in an Eirich mixer (EL-5 Profi Plus). An aqueous solution of nitric acid (520 ml, 15 wt. %) is added to the mixer and mixed for about 9 minutes. The obtained blend is aged at about 25° C. for about 1 hour and then formed into cylindrical extrudates (3.5 mm diameter) using an ETP1 Bonnot lab extruder, dried at 70° C. followed by 120° C. for about 12 hours, calcined at 600° C. for 2 hours in air in a muffle furnace, and cooled to room temperature without external cooling.

About 73 grams of the calcined alumina extrudates are impregnated utilizing incipient wetness techniques. The calcined alumina extrudates are brought into contact with an aqueous solution containing 29.7 grams of chromium(VI) oxide, 1 gram of lanthanum oxide and 2.5 grams of sodium dichromate dihydrate. The wet extrudates are aged at about 25° C. for about 12 hours in a closed container. The sample is then dried for about 6 hours at 120° C. and calcined at 750° C. for 2 hours in air in a muffle furnace and cooled to room temperature without external cooling. The resulting catalyst has a composition of 24.24 wt. % Cr₂O₃, 0.53 wt. % Na₂O, 1.02 wt. % La₂O₃, and 74.21 wt. % Al₂O₃.

### Example 2

The alumina support with 95 wt. % eta-alumina and 5 wt. % gamma-alumina used for the catalyst preparation is prepared as follows: 2969 grams of Bayerite (Pural BT, SASOL) and 139 grams of pseudoboehmite (PBAM-05, Chika Pvt. Ltd.) mixed for 10 minutes in an Eirich mixer (EL-5 Profi Plus). An aqueous solution of nitric acid (520 ml, 15 wt. %) is added to the mixer and mixed for about 8.5 minutes. The obtained blend is aged at about 25° C. for about 1 hour and then formed into cylindrical extrudates (3.5 mm diameter) using an ETP1 Bonnot lab extruder. These extrudates are dried at 70° C. followed by 120° C. for about 12 hours. These extrudates are calcined at 600° C. for 2 hours in air in a muffle furnace and cooled to room temperature without external cooling.

About 73 grams of the calcined alumina extrudates are impregnated utilizing incipient wetness techniques. The calcined alumina extrudates are brought into contact with an aqueous solution containing 29.7 grams of chromium(VI) oxide, 1 gram of lanthanum oxide and 2.5 grams of sodium dichromate dihydrate. The wet extrudates are aged at about 25° C. for about 12 hours in a closed container. The sample is then dried for about 6 hours at 120° C. and calcined at 750° C. for 2 hours in air in a muffle furnace and cooled to room temperature without external cooling. The resulting catalyst has a composition of 24.24 wt. % Cr₂O₃, 0.53 wt. % Na₂O, 1.02 wt. % La₂O₃, and 74.21 wt. % Al₂O₃.

### Example 3

The alumina support with 90 wt. % eta-alumina and 10 wt. % gamma-alumina used for the catalyst preparation is prepared as follows: 2813 grams of Bayerite (Pural BT, SASOL) and 277 grams of pseudoboehmite (PBAM-05, Chika Pvt. Ltd.) mixed for 10 minutes in an Eirich mixer (EL-5 Profi Plus). An aqueous solution of nitric acid (520 ml, 15 wt. %) is added to the mixer and mixed for about 8.5 minutes. The obtained blend is aged at about 25° C. for about 1 hour and then formed into cylindrical extrudates (3.5 mm diameter) using an ETP1 Bonnot lab extruder. These extrudates are dried at 70° C. followed by 120° C. for about 12 hours. These extrudates are calcined at 600° C. for 2 hours in air in a muffle furnace and cooled to room temperature without external cooling.

About 73 grams of the calcined alumina extrudates are impregnated utilizing incipient wetness techniques. The calcined alumina extrudates are brought into contact with an aqueous solution containing 29.7 grams of chromium(VI) oxide, 1 gram of lanthanum oxide and 2.5 grams of sodium dichromate dihydrate. The wet extrudates are aged at about 25° C. for about 12 hours in a closed container. The sample is then dried for about 6 hours at 120° C. and calcined at 750° C. for 2 hours in air in a muffle furnace and cooled to room temperature without external cooling. The resulting catalyst has a composition of 24.24 wt. % Cr₂O₃, 0.53 wt. % Na₂O, 1.02 wt. % La₂O₃, and 74.21 wt. % Al₂O₃.

### Example 4

The alumina support with 85 wt. % eta-alumina and 15 wt. % gamma-alumina used for the catalyst preparation is prepared as follows: 2656 grams of Bayerite (Pural BT, SASOL) and 416 grams of pseudoboehmite (PBAM-05, Chika Pvt. Ltd.) mixed for 10 minutes in an Eirich mixer (EL-5 Profi Plus). An aqueous solution of nitric acid (520 ml, 15 wt. %) is added to the mixer and mixed for about 6 minutes. The obtained blend is aged at about 25° C. for about 1 hour and then formed into cylindrical extrudates (3.5 mm diameter) using an ETP1 Bonnot lab extruder. These extrudates are dried at 70° C. followed by 120° C. for about 12 hours. These extrudates are calcined at 600° C. for 2 hours in air in a muffle furnace and cooled to room temperature without external cooling.

About 73 grams of calcined alumina extrudates prepared are impregnated to incipient wetness with an aqueous solution containing 29.7 grams of chromium(VI) oxide, 1 gram of lanthanum oxide and 2.5 grams of sodium dichromate dihydrate. The wet extrudates are aged at about 25° C. for about 12 hours in a closed container. The sample is then dried for about 6 hours at 120° C. and calcined at 750° C. for 2 hours in air in a muffle furnace and cooled to room temperature without external cooling. The resulting catalyst has a composition of 24.24 wt. % Cr₂O₃, 0.53 wt. % Na₂O, 1.02 wt. % La₂O₃, and 74.21 wt. % Al₂O₃.

### Example 5

The alumina support with 80 wt. % eta-alumina and 20 wt. % gamma-alumina used for the catalyst preparation is prepared as follows: 2500 grams of Bayerite (Pural BT, SASOL) and 554 grams of pseudoboehmite (PBAM-05, Chika Pvt. Ltd.) mixed for 10 minutes in an Eirich mixer (EL-5 Profi Plus). An aqueous solution of nitric acid (500 ml, 10 wt. % HNO₃+20 ml water) is added to the mixer and mixed for about 12.5 minutes. The obtained blend is aged at about 25° C. for about 1 hour and then formed into cylindrical extrudates (3.5 mm diameter) using an ETP1 Bonnot lab extruder. These extrudates are dried at 70° C. followed by 120° C. for about 12 hours. These extrudates are calcined at 600° C. for 2 hours in air in a muffle furnace and cooled to room temperature without external cooling.

About 73 grams of calcined alumina extrudates prepared are impregnated utilizing incipient wetness techniques. The calcined alumina extrudates are brought into contact with an aqueous solution containing 29.7 grams of chromium(VI) oxide, 1 gram of lanthanum oxide. and 2.5 grams of sodium dichromate dihydrate. The wet extrudates are aged at about 25° C. for about 12 hours in a closed container. The sample is then dried for about 6 hours at 120° C. and calcined at 750° C. for 2 hours in air in a muffle furnace and cooled to room temperature without external cooling. The resulting catalyst has a composition of 24.24 wt. % Cr₂O₃, 0.53 wt. % Na₂O, 1.02 wt. % La₂O₃, and 74.21 wt. % Al₂O₃.

### Example 6

The alumina support with pure gamma-alumina used for the catalyst preparation was prepared as follows: 2000 g of pseudoboehmite (PBAM-05, Chika Pvt. Ltd.) was mixed for 10 minutes in an Eirich mixer (EL-5 Profi Plus). An aqueous solution of nitric acid (1290 ml, 1.5 wt. %) was added to the mixer and mixed for about 18 minutes. The obtained blend was aged at about 25° C. for about 1 hour and then formed into cylindrical extrudates (3.5 mm diameter) using an ETP1 Bonnot lab extruder. The catalyst extrudates were dried at 70° C. followed by 120° C. for about 12 hours and calcined at 600° C. for 2 hours in air in a muffle furnace and cooled to room temperature without external cooling.

About 73 grams of the calcined alumina extrudates were impregnated utilizing incipient wetness techniques. The calcined alumina extrudates are brought into contact with an aqueous solution containing 29.7 grams of chromium(VI) oxide, 1 gram of lanthanum oxide. and 2.5 grams of sodium dichromate dihydrate. The wet extrudates were aged at about 25° C. for about 12 hours in a closed container. The sample was then dried for about 6 hours at 120° C. and calcined at 750° C. for 2 hours in air in a muffle furnace and cooled to room temperature without external cooling.

The resulting catalyst has a composition of 24.24 wt. % Cr₂O₃, 0.53 wt. % Na₂O, 1.02 wt. % La₂O₃, and 74.21 wt. % Al₂O₃.

### Example 7—Catalyst Testing

The dehydrogenation activity of the prepared catalyst is measured in a tubular fixed-bed quartz reactor under atmospheric pressure. Catalyst loading and reactor details were as follows: Catalyst weight=8.5 g, catalyst particle size=3-3.2 mm, inert quartz weight=8.5 g, inert quartz chips=0.4-0.5 mm, reactor ID=16 mm, reactor OD=19 mm. Catalyst and inert quartz were divided into equal parts by weight and then loaded into the reactor by mixing catalyst and inert. Isobutane (99.9 vol. %) was used as the feed. Quartz chips having a size of 1-1.4 mm were loaded above the catalyst bed. A nitrogen purge was employed between the steps of dehydrogenation, catalyst regeneration/oxidation, and reduction with hydrogen. The total feed flow in the dehydrogenation step corresponded to a gas hourly space velocity (GHSV) of 600 ml h⁻¹ g⁻¹. The reactor outlet gases were analyzed by an online gas chromatograph (Agilent 6890) equipped with a flame ionization detector for hydrocarbon analysis and a thermal conductivity detector for hydrogen analysis. The reactant and product flow rates were measured using a Ritter type wet gas flow meter. The reactor was operated at atmospheric pressure and in a cyclic mode with the following steps: 1) catalyst oxidation with air with a start temperature of 650° C. for 20 min.; 2) purge the catalyst with nitrogen at 650° C. for 3 min.; 3) reduce the catalyst with H₂ with a start temperature of 650° C. for 6 min.; 4) cool under nitrogen from 650° C. to 585° C. and maintain a temperature of 585° C. for 30 min.; 5) dehydrogenation of isobutane with a start temperature of 585° C. for 21 min.; 6) analyze the reactor outlet gas composition with gas chromatograph (GC) at the 20th minute from the start of the isobutane feed. Steps 1 to 6 were repeated 20 times. The catalyst performance data (10 cycle average) after catalyst stabilization is given in Table 1.

### Example 8—Catalyst Stability Evaluation

Catalyst stability evaluation was carried out by an artificial accelerated aging procedure in a cyclic mode of operation. The cycle included H₂—N₂-isobutane-N₂-air flow stages with different time durations. The aging was carried out at 820° C. for 72 hours. Catalyst stability evaluation parameters included a catalyst weight of 8.5 grams, isobutane being supplied at a GHSV of 400 ml h⁻¹ g⁻¹ and an air to isobutane volume ratio of 2. The catalyst was oxidized under air for 15 minutes, and then subjected to purging with nitrogen for 3 minutes. The catalyst was then reduced with H₂ for 6 minutes and then subjected to purging with nitrogen for 3 minutes. The catalyst was then subject to a isobutane flow for 3 minutes and then subjected to purging with nitrogen for 3 minutes. After aging, the performance of the catalyst was evaluated under the cyclic mode previously described. The products from the dehydrogenation of isobutane were analyzed with a gas chromatograph.

The surface area and crush strength of catalysts were analyzed. The results show that the catalyst prepared using alumina support containing eta-alumina and gamma-alumina shows similar surface area. The catalyst prepared using eta-alumina show lower surface area whereas the catalyst prepared using gamma-alumina shows high surface area. The catalyst prepared using eta-alumina as the carrier shows lower crush strength and the crush strength increases with the increase in the gamma-alumina content in the carrier used for the preparation of the catalyst.

The results from Table 1 show that similar isobutylene yield for catalyst prepared using alumina support containing up to 15 wt. % gamma-alumina. The catalyst with alumina support having 20 wt. % gamma-alumina shows lower isobutylene yield and lower catalyst stability.

TABLE 1

| | Conversion (mol %) | | | | Selectivity (mol %) | | | | Yield (mol %) | | | |
|---|---|---|---|---|---|---|---|---|---|---|---|---|
| | Before | | After | | Before | | After | | Before | | After | |
| | aging | | aging | | aging | | aging | | aging | | aging | |
| Example | Aver. | SD | Aver. | SD | Aver. | SD | Aver. | SD | Aver. | SD | Aver. | SD |
| 1 | 54.8 | 0.4 | 45.0 | 0.6 | 94.0 | 0.9 | 91.9 | 1.1 | 51.5 | 0.5 | 41.4 | 0.5 |
| 2 | 54.4 | 0.4 | 43.0 | 0.3 | 91.4 | 1.2 | 92.2 | 1.3 | 49.8 | 0.4 | 39.6 | 0.3 |
| 4 | 55.6 | 0.9 | 43.5 | 0.5 | 92.4 | 1.8 | 91.6 | 1.8 | 51.4 | 0.4 | 39.9 | 0.5 |
| 5 | 51.6 | 0.4 | 40.2 | 0.5 | 93.3 | 1.6 | 91.5 | 1.4 | 48.2 | 0.5 | 36.8 | 0.5 |
| 6 | 43.2 | 0.6 | | | 92.1 | 1.7 | | | 39.9 | 0.4 | | |

When ranges are disclosed herein, ranges from any lower limit may be combined with any upper limit to recite a range not explicitly recited, as well as, ranges from any lower limit may be combined with any other lower limit to recite a range not explicitly recited. In the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing detailed description and examples. It should be understood, however, that the detailed description and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein.

1. A method for making an alkane dehydrogenation catalyst, the method comprising:
providing a transition alumina support derived from a plurality of aluminium hydroxides, the plurality of aluminium hydroxides containing about 85 weight percent (wt. %) to about 99 wt. % of crystalline aluminium trihydroxide and about 1 wt. % to about 15 wt. % of crystalline aluminium oxide-hydroxide or gelatinous aluminium hydroxide or combinations thereof;
contacting the alumina support with a water-soluble chromium(III) oxide source and an alkali metal oxide source to provide an impregnated alumina support; and
drying and calcining the impregnated alumina support to produce an alkane dehydrogenation catalyst containing about 60 wt. % to about 95 wt. % of alumina, about 5 wt. % to about 40 wt. % of the chromium(III) oxide, and about 0.1 wt. % to about 5 wt. % of the alkali metal oxide.

2. The method of claim 1, wherein the transition alumina support is substantially an eta-alumina.

3. The method of claim 1, wherein the alkane dehydrogenation catalyst contains about 60 wt. % to about 90 wt. % of alumina, about 10 wt. % to about 40 wt. % of the chromium(III) oxide, and about 0.1 wt. % to about 5 wt. % of the alkali metal oxide.

4. The method of claim 1, wherein the crystalline aluminium trihydroxide contains one or more of bayerite and nordstrandite.

5. The method of claim 1, wherein the crystalline aluminium oxide-hydroxide contains boehmite.

6. The method of claim 1, wherein the gelatinous aluminium hydroxide contains one or more of amorphous aluminium hydroxide and pseudoboehmite.

7. The method of claim 1, wherein the impregnated alumina support is calcined at a temperature ranging from about 700 degrees Celsius (° C.) to 800° C.

8. A method for dehydrogenation of an alkane, the method comprising:
loading a reactor with a dehydrogenation catalyst produced by:
providing a transition alumina support derived from a plurality of aluminium hydroxides, the plurality of aluminium hydroxides containing about 85 weight percent (wt. %) to about 99 wt. % of crystalline aluminium trihydroxide and about 1 wt. % to about 15 wt. % of crystalline aluminium oxide-hydroxide or gelatinous aluminium hydroxide or combinations thereof;
contacting the alumina support with a water-soluble chromium(III) oxide source and an alkali metal oxide source to provide an impregnated alumina support; and
drying and calcining the impregnated alumina support to produce an alkane dehydrogenation catalyst containing about 60 wt. % to about 95 wt. % of alumina, about 5 wt. % to about 40 wt. % of the chromium(III) oxide, and about 0.1 wt. % to about 5 wt. % of the alkali metal oxide; and
supplying a feed containing an alkane through the reactor at a temperature sufficient to dehydrogenate the alkane and produce an olefin.

9. The method of claim 8, wherein the transition alumina support is substantially an eta-alumina.

10. The method of claim 9, wherein the crystalline aluminium trihydroxide contains one or more of bayerite and nordstrandite.

11. The method of claim 8, wherein the crystalline aluminium oxide-hydroxide contains boehmite.

12. The method of claim 8, wherein the gelatinous aluminium hydroxide contains one or more of amorphous aluminium hydroxide or pseudoboehmite.

13. The method of claim 8, wherein the impregnated alumina support is calcined at a temperature ranging from about 700° C. to 800° C.

14. The method of claim 8, wherein the temperature sufficient to dehydrogenate the alkane ranges from 400° C. to 800° C.
